(19) United States
(12) Patent Application Publication
(10) Pub. No.: US 2025/0276305 A1
(43) Pub. Date: Sep. 4, 2025

(54) CATALYST FOR THE PRODUCTION OF 1,3-BUTADIENE GIVING A HIGH YIELD BASED ON A SUPPORT COMPRISING ALUMINIUM AND SODIUM

(71) Applicants: SYNTHOS S.A., OSWIECIM (PL); ECOVYST INC., Malvern, PA (US)

(72) Inventors: Jaroslaw ROGOZA, OSWIECIM (PL); Ewelina JANKOWIAK, OSWIECIM (PL); Magdalena SITKO, OSWIECIM (PL); Szymon SKOWRONEK, OSWIECIM (PL); Parag Rasiklal SHAH, Lafayette Hill, PA (US); Yatao Rachel HU, Malvern, PA (US)

(21) Appl. No.: 19/100,335

(22) PCT Filed: Aug. 1, 2023
(86) PCT No.: PCT/EP2023/071314

§ 371 Date: Jan. 31, 2025

(30) Foreign Application Priority Data
Aug. 2, 2022 (EP) 22461589.8

(51) Int. Cl. B01J 23/20; B01J 21/08; B01J 35/61; B01J 35/63; B01J 37/02; B01J 37/08; C01B 33/143; C01B 33/154; C01B 33/157; C07C 1/24

(52) U.S. Cl. CPC B01J 23/20; B01J 21/08; B01J 35/615; B01J 35/635; B01J 35/638; B01J 37/0201; B01J 37/0236; B01J 37/08; C01B 33/143; C01B 33/1546; C01B 33/157; C07C 1/24

(57) ABSTRACT

The present invention relates to a supported catalyst comprising a support and 0. 1 to 10 wt. % of tantalum, calculated as Ta₂O₅ and based on the total weight of the catalyst. wherein the supported catalyst further comprises from 50 to 350 ppm of aluminium and from 300 to 500 ppm of sodium, based on the total weight of the catalyst, respectively. Moreover, the invention relates to a catalyst reaction tube for the production of 1,3-butadiene comprising at least one packing of the supported catalyst as defined herein, to a reactor for the production of 1,3-butadiene comprising one or more of the catalyst reaction tubes as defined herein, and to a plant for the production of 1,3-butadiene comprising one or more of the reactors as defined herein. The invention also relates to a process for the production of 1,3-butadiene as defined herein and to a process for the production of the supported catalyst as defined herein. Finally, the present invention relates to the use of the supported catalyst as defined herein for the production of 1,3-butadiene from a feed comprising ethanol and acetaldehyde and to the use of aluminium in an amount in a range of from 50 to 350 ppm in a supported catalyst for the production of 1,3-butadiene from a feed comprising ethanol and acetaldehyde for increasing the yield of 1,3-butadiene.

The present invention relates to a supported catalyst comprising a support and 0.1 to 10 wt. % of tantalum, calculated as Ta₂O₅ and based on the total weight of the catalyst, wherein the supported catalyst further comprises from 50 to 350 ppm of aluminium and from 300 to 500 ppm of sodium, based on the total weight of the catalyst, respectively. Moreover, the invention relates to a catalyst reaction tube for the production of 1,3-butadiene comprising at least one packing of the supported catalyst as defined herein, to a reactor for the production of 1,3-butadiene comprising one or more of the catalyst reaction tubes as defined herein, and to a plant for the production of 1,3-butadiene comprising one or more of the reactors as defined herein. The invention also relates to a process for the production of 1,3-butadiene as defined herein and to a process for the production of the supported catalyst as defined herein. Finally, the present invention relates to the use of the supported catalyst as defined herein for the production of 1,3-butadiene from a feed comprising ethanol and acetaldehyde and to the use of aluminium in an amount in a range of from 50 to 350 ppm in a supported catalyst for the production of 1,3-butadiene from a feed comprising ethanol and acetaldehyde for increasing the yield of 1,3-butadiene.

1,3-Butadiene is one of the most important raw materials in the synthetic rubber industry, where it is used as a monomer in the production of a wide range of synthetic polymers, such as polybutadiene rubbers, acrylonitrile-butadiene-styrene polymers, styrene-butadiene rubbers, nitrile-butadiene rubbers, and styrene-butadiene latexes. 1,3-Butadiene is, for example, obtained as a by-product of ethylene manufacturing in naphtha steam cracking and can be isolated by extractive distillation (Chem. Soc. Rev., 2014, 43, 7917; ChemSusChem, 2013, 6, 1595; Chem. Central J., 2014, 8, 53).

The depletion of non-renewable, fossil fuels-derived resources as well as environmental considerations have recently become strong driving forces for the exploration of renewable sources of 1,3-butadiene and its precursors. Of the wide range of the available renewable sources, biomass seems to have the greatest potential in the context of use for the production of 1,3-butadiene. This strategy has two main advantages: independence from fossil fuels and reduction of CO₂ emissions (ChemSusChem, 2013, 6, 1595).

The conversion of ethanol, obtainable e.g. from biomass, to 1,3-butadiene may be performed in two ways reported in the literature: as one-step process (Lebedev process) and as two-step process (Ostromislensky process).

The one-step process, reported by Lebedev in the early part of the 20^{th} century, is carried out by direct conversion of ethanol to 1,3-butadiene, using multifunctional catalysts tuned with acid-base properties (J. Gen. Chem., 1933, 3, 698; Chem. Ztg., 1936, 60, 313).

On the other hand, the so-called two-step process may be performed by converting, in a first step, ethanol to acetaldehyde. The aim of this first step is to feed a second step or reactor with such mixture of ethanol and acetaldehyde. In the second step, conversion of the mixture of ethanol and acetaldehyde to 1,3-butadiene over, for example, a silica-supported tantalum catalyst takes place (Catal. Today, 2016, 259, 446).

US 2018/0208522 A1 relates to a catalyst for the conversion of a feed comprising ethanol and acetaldehyde to 1,3-butadiene. The catalyst comprises at least the element tantalum, and at least one mesoporous oxide matrix that has undergone an acid wash comprising at least 90% by weight of silica before washing, the mass of the element tantalum being in the range 0.1% to 30% of the mass of said mesoporous oxide matrix. The teaching of US 2018/0208522 A1 relies on acid washing of the mesoporous oxide support for increasing the selectivity of the catalyst towards 1,3-butadiene and/or the productivity of the catalyst towards 1,3-butadiene. At the end of the washing step and before impregnation of the active element(s), the catalyst contains amounts of sodium in the range of 0 to 500 ppm. Concentrations of aluminium in the catalysts and yields of 1,3-butadiene are not disclosed in US 2018/0208522 A1.

WO 2020/126920 A1 relates to a method for producing 1,3-butadiene from ethanol, in two reaction steps, comprising a step a) of converting the ethanol into acetaldehyde and a step b) of conversion into 1,3-butadiene, the step b) simultaneously implementing a reaction step and a regeneration step in (n+n/2) fixed-bed reactors, n being equal to 4 or to a multiple thereof, comprising a catalyst, said regeneration step comprising four consecutive regeneration phases, the step b) also implementing three regeneration loops.

US 2018/200694 A1 relates to a mesoporous mixed oxide catalyst that comprises silicon and at least one metal M that is selected from the group that consists of the elements of groups 4 and 5 of the periodic table and mixtures thereof, with the mass of metal M being between 0.1 and 20% of the mixed oxide mass.

WO 2022/165190 A1 relates to a method for making a supported tantalum oxide catalyst precursor or catalyst with controlled tantalum distribution and the resulting supported tantalum catalyst. In an embodiment, the method comprises selecting a tantalum precursor with appropriate reactivity with the surface hydroxyls of the solid oxide support material to give a desired tantalum distribution in the catalyst precursor or catalyst. In another embodiment, the method comprises controlling the number of surface hydroxyls available on the support material to react with the tantalum precursor by thermal methods, such as calcining, to achieve the desired tantalum distribution.

Thus, there is an ongoing need for the provision of catalysts for the production of 1,3-butadiene that show high activity and are able to provide high yields of 1,3-butadiene.

A supported catalyst comprising or consisting of
(i) a support, and
(ii) 0.1 to 10 wt %, preferably 2 to 4 wt. %, of tantalum calculated as Ta₂O₅ and based on the total weight of the catalyst,
   wherein the supported catalyst further comprises from 50 to 350 ppm, preferably from 75 to 350 ppm, more preferably from 100 to 350 ppm, more preferably from 100 to 300 ppm, more preferably from 100 to 275 ppm, most preferably from 150 to 250 ppm, of aluminium, based on the total weight of the catalyst, and from 300 to 500 ppm, preferably from 350 to 500 ppm, more preferably from 380 to 500 ppm, more preferably from 380 to 450 ppm, most preferably from 380 to 410 ppm, of sodium, based on the total weight of the catalyst.

During the studies underlying the present invention, it was found that an increase in sodium level in the catalysts compared to the sodium level of an ultra pure catalyst comprising 6 ppm of aluminium and 31 ppm of sodium (based on the total weight of the catalyst, respectively) increases the total conversion, however, it has a strongly detrimental effect on the selectivity to 1,3-butadiene. Thus, both the achieved 1,3-butadiene yield and productivity of 1,3-butadiene are reduced if only the sodium level is increased in the catalyst. However, it was surprisingly found that a simultaneous increase in aluminium level offsets the detrimental effects of the increased sodium level in the catalyst. In the catalytic tests of the catalysts according to the invention, not only the total conversion was increased, but also the selectivity loss to 1,3-butadiene was small, so that overall an increase in both yield and productivity of 1,3-butadiene was advantageously achieved (cf. Table 2 and examples below).

Sodium and aluminium levels as indicated herein in parts per million relate to the total weight of the supported catalyst including tantalum as tantalum oxide. The same applies to the tantalum levels as indicated herein in wt. %.

In one preferred embodiment, the support of the supported catalyst according to the invention comprises one or more of ordered and non-ordered porous silica supports, other porous oxide supports and mixtures thereof, preferably from ZrO₂, TiO₂, MgO, ZnO, NiO, and CeO₂.

Most preferably, the support of the supported catalyst according to the invention is a silica support, preferably an ordered or non-ordered porous silica support.

Supported catalysts are particularly advantageous, because they allow control of the concentration and dispersion of the active sites, simple preparation of the catalyst by impregnation of any form and shape of the support, and easy access of the reacting molecules to all active sites of the catalyst.

Preferably, the supported catalyst according to the invention has a BET specific surface area in a range of from 130-550 m²/g, preferably in a range of from 190 to 280 m²/g.

Preferably, the supported catalyst according to the invention has an average pore diameter in a range of from 30 to 300 Å.

Preferably, the supported catalyst according to the invention has a pore volume in a range of from 0.2 to 1.5 cm³/g.

Surface area (SA) and pore volume (PV) were measured by Nitrogen Porosimetry using an Autosorb-6 Testing Unit from Quantachrome Corporation (now Anton Paar GmbH). Samples were first degassed at 350° C. for at least 4 hours on the Autosorb-6 Degassing Unit. A multipoint surface area is calculated using the BET theory taking data points in the P/P₀ range 0.05 to 0.30. A pore volume measurement is recorded at P/P₀ of 0.984 on the desorption leg. Average pore diameter is calculated using the following equation assuming cylindrical pores:

$$Average pore diameter ( Å ) = \frac{40000 \times PV ( \frac{cc}{g} )}{SA ( \frac{{m}^{2}}{g} )}$$

According to a preferred embodiment of the invention, the weight ratio of aluminium to sodium in the supported catalyst is in a range of from 0.1 to 1.2, preferably 0.2 to 1.0, more preferably 0.3 to 0.8, most preferably 0.4 to 0.7.

In a second aspect, the present invention relates to a catalyst reaction tube for the production of 1,3-butadiene comprising at least one packing of the supported catalyst according to the invention and one or more packings of inert material.

Preferably, the inert material is selected from the group consisting of silicon carbide, inert ceramic beds, ceramic beads, extrudates, rings with a diameter of 2-7 mm, stainless steel mesh, foams, and mixtures thereof.

According to a preferred embodiment, the packings of the inert material contact and separate the packings of the supported catalyst according to the invention, i.e. the reaction zones, from one another (if more than one packing of the supported catalyst is present in the catalyst reaction tube). They are preferably located at the reactant feed inlet and outlet of the reaction tube.

According to one embodiment, the catalyst reaction tube is loaded with one packing of the supported catalyst according to the invention, preferably in the centre of the catalyst reaction tube. The supported catalyst according to the invention is in contact with a packing of inert material on either side, i.e. the packings of inert material are preferably located at the feed inlet and outlet of the catalyst reaction tube. According to this embodiment, the catalyst reaction tube comprises one reaction zone.

According to another embodiment, the catalyst reaction tube is loaded alternatingly with packings of the supported catalyst according to the invention and packings of inert material. The packings of inert material are preferably located at the feed inlet and outlet of the catalyst reaction tube and contact the packings of the supported catalyst according to the invention. According to this embodiment, the catalyst reaction tube comprises more than one reaction zone.

In a third aspect, the present invention relates to a reactor for the production of 1,3-butadiene comprising one or more of the catalyst reaction tubes according to the invention.

In a fourth aspect, the present invention relates to a plant for the production of 1,3-butadiene comprising one or more of the reactors as defined herein, and means for regenerating the supported catalyst in said one or more reactors, preferably wherein the plant also comprises an acetaldehyde-producing pre-reactor with one or more reaction tubes comprising a supported or unsupported (bulk) catalyst comprising one or more of zinc, copper, silver, chromium, magnesium and nickel, preferably comprising one or more of zinc and copper.

Tantalum oxide, as contained in the supported catalyst according to the invention, is inactive in the oxidation of ethanol to acetaldehyde. Thus, in order to produce 1,3-butadiene with the supported catalyst according to the invention, the feed stream has to contain ethanol and acetaldehyde. This mixture of ethanol and acetaldehyde can, for instance, be produced in the plant from ethanol in an acetaldehyde-producing pre-reactor comprising a supported or unsupported (bulk) catalyst as defined above, and then be fed into a reactor for the production of 1,3-butadiene comprising one or more of the catalyst reaction tubes according to the invention. Alternatively, ethanol and acetaldehyde can be obtained from commercial sources and fed directly into a reactor for the production of 1,3-butadiene comprising one or more of the catalyst reaction tubes according to the invention.

In a fifth aspect, the present invention relates to a process for the production of 1,3-butadiene, the process comprising
(i) contacting a feed comprising ethanol and acetaldehyde with the supported catalyst according to the invention to obtain a raw product comprising 1,3-butadiene.

Preferably, in the process according to the invention, the (i) contacting takes place at a temperature in a range of from 200 to 500° C., preferably from 250 to 450° C., more preferably from 300 to 400° C.

In a preferred embodiment of the process according to the invention, the (i) contacting takes place at a weight hourly space velocity in a range of from 0.2 to 10 h⁻¹, preferably from 1 to 7 h⁻¹, more preferably from 2 to 5 h⁻¹.

Preferably, the (i) contacting takes place at a pressure in a range of from 0 to 10 barg, preferably from 1 to 3 barg, most preferably from 1 to 2 barg.

Preferably, the process according to the invention further comprises the following step(s):
(ii) separating the raw product at least into a first portion comprising 1,3-butadiene, a second portion comprising acetaldehyde and a third portion comprising ethanol,
preferably wherein at least part of the second, of the third, or of both the second and of the third portions is recycled into the feed.

According to a preferred embodiment of the process according to the invention, the (i) contacting takes place in a continuous flow of the feed in a reactor as defined herein.

According to another preferred embodiment of the process according to the invention, the feed comprises at least 50 wt. % of ethanol, preferably comprises 60 to 75 wt. % of ethanol, based on the total weight of the feed.

According to another preferred embodiment of the process according to the invention, the feed comprises at least 15 wt. % of acetaldehyde, preferably comprises 20 to 35 wt. % of acetaldehyde, based on the total weight of the feed.

According to another preferred embodiment of the process according to the invention, the molar ratio of ethanol to acetaldehyde in the feed is in a range of from 1 to 7, preferably of from 1.5 to 5, more preferably of from 1.7 to 4, most preferably of from 2.0 to 3.0.

In a sixth aspect, the present invention relates to a process for the production of the supported catalyst according to the invention comprising or consisting of the following steps:
(i) impregnation of the support with aluminium and sodium levels defined by the formulas below based on the weight of the catalyst support, with a solution of a tantalum precursor, to form a supported tantalum catalyst precursor,
   wherein the lower limit is defined by: Support [M]_{LL}=Catalyst [M]_{LL}/(1−Catalyst [Ta₂O₅] wt. %), with M=Na or Al; where Catalyst [Na]_{LL}=300 ppm and Catalyst [Al]_{LL}=50 ppm; and
   the upper limit is defined by: Support [M]_{UL}=Catalyst [M]_{UL}/(1−Catalyst [Ta₂O₅] wt. %), with M=Na or Al; where Catalyst [Na]_{UL}=500 ppm and Catalyst [Al]_{UL}=350 ppm;
(ii) drying the supported tantalum catalyst precursor, and
(iii) calcining the dried supported tantalum catalyst precursor, to form a supported tantalum catalyst.

In above formulae, Support [M]_{LL} designates the lower limit of the concentration (wt./wt.) of metals M (M being sodium or aluminium, respectively) in the support to be used and to be impregnated in step (i), which is dependent on
a. Catalyst [M]_{LL}, the lower limit of the concentration (wt./wt.) of metals M (M being sodium or aluminium, respectively) in the supported catalyst according to the invention to be ultimately obtained in step (iii), and
b. Catalyst [Ta₂O₅] wt. %, the concentration (wt./wt.) of Ta₂O₅ in the supported catalyst according to the invention to be ultimately obtained in step (iii).

Likewise, in above formulae, Support [M]_{UL} designates the upper limit of the concentration (wt./wt.) of metals M (M being sodium or aluminium, respectively) in the support to be used and to be impregnated in step (i), which is dependent on
a. Catalyst [M]_{UL}, the upper limit of the concentration (wt./wt.) of metals M (M being sodium or aluminium, respectively) in the supported catalyst according to the invention to be ultimately obtained in step (iii), and
b. Catalyst [Ta₂O₅] wt. %, the concentration (wt./wt.) of Ta₂O₅ in the supported catalyst according to the invention to be ultimately obtained in step (iii).

Preferred embodiments in terms of sodium and aluminium contents of the supported catalyst according to the first aspect of the present invention correspond to preferred embodiments regarding Catalyst [M]_{LL} and Catalyst [M]_{UL} regarding of the sixth aspect of the invention.

In one preferred embodiment, the support impregnated in step (i) of the process according to the invention comprises one or more of ordered and non-ordered porous silica, other porous oxides and mixtures thereof, preferably from ZrO₂, TiO₂, MgO, ZnO, NiO, and CeO₂.

Preferably, the support impregnated in step (i) of the process according to the invention is a silica support, preferably an ordered or non-ordered porous silica support.

According to a preferred embodiment of the process for the production of the supported catalyst according to the invention, the supported catalyst is a silica supported catalyst and the method comprises or consists of:
(i) reacting an aqueous silicate, preferably sodium silicate, solution with an acid, to form a hydrosol,
(ii) dispersion, preferably by means of spraying, more preferably by means of spraying into air and breaking into droplets, and gelation of the hydrosol, to form hydrogel beads,
(iii) one or more optional additional steps of (pre-)aging, acidification, washing and pH adjustment,
   a. aging of the hydrogel beads at temperature T1,
   b. acidification of the aged hydrogel beads,
   c. washing, preferably with water that is deionized and acidified to pH 3-4, of the acidified aged hydrogel beads,
   d. adjusting the pH of the washed hydrogel beads obtained in step (c), preferably to a pH of about 8-10,
(iv) aging of the hydrogel beads at temperature T2, with T2>T1 (if applicable, e.g. if one of the optional steps in (iii) are used),
(v) acidification of the aged hydrogel beads (obtained in step (iv)),
(vi) washing, preferably with water that is deionized and acidified to pH 3-4, of the acidified aged hydrogel beads (obtained in step (v)),
(vii) optionally adjusting the pH of the washed hydrogel beads obtained in step (vi), preferably to a pH in a range of about 3 to 10, most preferably to a pH of about 9,
(viii) drying the washed hydrogel beads obtained in step (vi) or (vii) to obtain a silica support, preferably by using an oven,
(ix) optionally, sieving of the silica support obtained in step (viii) (to collect the desired particle size fraction),
(x) impregnation of the silica support obtained in step (viii) or (ix) with a solution of a tantalum precursor, to form a supported tantalum catalyst precursor, preferably wherein the tantalum precursor is tantalum ethoxide, most preferably wherein the tantalum ethoxide precursor is stabilized with 2,4-pentanedione and/or dissolved in a suitable organic solvent such as isopropanol,
(xi) drying the supported tantalum catalyst precursor, preferably by heating at atmospheric pressure or under vacuum, and
(xii) calcining the dried supported tantalum catalyst precursor, preferably at a temperature of about 400 to 600° C. for about 2 to 5 hours, to form a supported tantalum catalyst.

As used herein, a “supported tantalum catalyst precursor” refers to an intermediate product, e.g., before calcination. In contrast, a “supported tantalum catalyst” is the product after calcination.

Preferably, temperature T1 in the process according to the invention is in a range of from 20 to 50° C.

Preferably, temperature T2 in the process according to the invention is in a range of from 40 to 100° C.

In a seventh aspect, the present invention relates to the use of the supported catalyst according to the invention for the production of 1,3-butadiene from a feed comprising ethanol and acetaldehyde, preferably for increasing the yield of 1,3-butadiene.

In an eighth aspect, the present invention relates to the use of aluminium in an amount in a range of from 50 to 350 ppm, preferably from 75 to 350 ppm, more preferably from 100 to 350 ppm, more preferably from 100 to 300 ppm, more preferably from 100 to 275 ppm, most preferably from 150 to 250 ppm, based on the total weight of the catalyst, in a supported catalyst for the production of 1,3-butadiene from a feed comprising ethanol and acetaldehyde, the catalyst comprising or consisting of
a support,
300 to 500 ppm, preferably 350 to 500 ppm, more preferably 380 to 500 ppm, more preferably 380 to 450 ppm, most preferably 380 to 410 ppm, of sodium, based on the total weight of the catalyst, and
0.1 to 10 wt. %, preferably 2 to 4 wt. %, of tantalum, calculated as Ta₂O₅ and based on the total weight of the catalyst,
   for increasing the yield of 1,3-butadiene.

In a ninth aspect, the present invention relates to the use of sodium in an amount in a range of from 300 to 500 ppm, preferably from 350 to 500 ppm, more preferably from 380 to 500 ppm, more preferably from 380 to 450 ppm, most preferably from 380 to 410 ppm, based on the total weight of the catalyst, in a supported catalyst for the production of 1,3-butadiene from a feed comprising ethanol and acetaldehyde, the catalyst comprising or consisting of
a support,
50 to 350 ppm, preferably 75 to 350 ppm, more preferably from 100 to 350 ppm, more preferably 100 to 300 ppm, more preferably 100 to 275 ppm, most preferably 150 to 250 ppm, of aluminium, based on the total weight of the catalyst, and
0.1 to 10 wt. %, preferably 2 to 4 wt. %, of tantalum, calculated as Ta₂O₅ and based on the total weight of the catalyst,
   for increasing the yield of 1,3-butadiene.

In a tenth aspect, the present invention relates to the use of sodium in an amount in a range of from 300 to 500 ppm, preferably from 350 to 500 ppm, more preferably from 380 to 500 ppm, more preferably from 380 to 450 ppm, most preferably from 380 to 410 ppm, and of aluminium in an amount in a range of from 50 to 350 ppm, preferably from 75 to 350 ppm, more preferably from 100 to 350 ppm, more preferably from 100 to 300 ppm, more preferably from 100 to 275 ppm, most preferably from 150 to 250 ppm, based on the total weight of the catalyst respectively, in a supported catalyst for the production of 1,3-butadiene from a feed comprising ethanol and acetaldehyde, the catalyst comprising or consisting of
a support,
0.1 to 10 wt. %, preferably 2 to 4 wt. %, of tantalum, calculated as Ta₂O₅ and based on the total weight of the catalyst,
   for increasing the yield of 1,3-butadiene.

Preferred embodiments of a certain aspect of the present invention (cf. aspects one to ten above) correspond to or can be derived from preferred embodiments of the other aspects of the invention (as defined above), respectively, as long as technically sensible.

### EXAMPLES

### 1. Silica Support Preparation

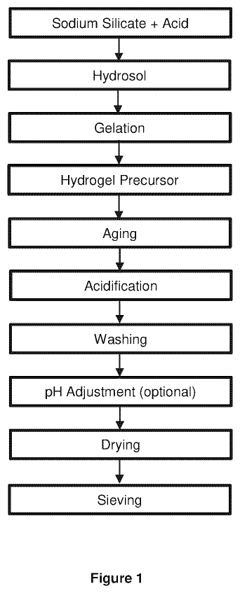

The following is a description of the general steps used for making the silica support according to an embodiment of the present disclosure. A flow chart showing the general steps used in making silica support according to an embodiment of the present disclosure is provided in FIG. 1. A more detailed description of the silica support and methods of making it are found in co-pending application number U.S patent application Ser. No. 16/804,610, which is herein incorporated by reference.

In one embodiment, a dilute sodium silicate solution of 3.3 weight ratio SiO₂:Na₂O was first reacted with dilute sulfuric acid, to form a hydrosol having the following composition: 12 wt. % SiO₂ and H₂SO₄:Na₂O in a molar ratio of 0.8. As a result, the resulting hydrosol was basic. In one embodiment, the sodium silicate solution contained approximately 250ppm aluminium on SiO₂ weight basis. In one embodiment, a higher purity silicate with low aluminium (<10 ppm on SiO₂ weight basis) was used to make silica with lower aluminium content.

The hydrosol was then sprayed into air, where it broke into droplets and solidified into beads having a diameter of several millimeters before it was caught in a solution such as water or a solution that buffers the pH of the beads/solution system at a basic pH of about 9 (such as aqueous solution of ammonium sulfate, sodium bicarbonate, etc.). Higher aging temperature and/or longer aging times reduces the silica surface area. Generally, for hydrogel caught in ammonium sulfate solution to achieve a surface area of about 300 m²/g, aging is conducted at 70° C. at pH about 9 for about 16 hours.

Acid was then added to lower the pH to about 2. The hydrogel beads were then washed with water that was acidified to a pH of about 3 to reduce sodium levels. The aged and washed hydrogel beads contain about 15-18% SiO₂. Once washed, the pH of the beads was increased to about 9 using ammonium hydroxide solution. The beads were then dried using an oven. Finally, the beads were sieved to get the desired particle size fraction. Note that pH adjustment before drying is optional, and beads are typically dried from pH 3-9.

In one embodiment, the described process can be modified to optionally include multiple aging steps at increasing temperatures with each aging step followed by acidification and washing steps to get the desired combination of surface area and sodium levels. In one embodiment, optionally, washing can be done before the aging step.

Following the procedure outlined above, one can obtain a silica gel bead with a surface area of about 230-300 m²/g, a pore volume of about 0.95-1.05 cm³/g, aluminium <500 ppm (depending on silicate purity and/or the process and conditions used to carry out the washing and aging steps), and sodium <1000 ppm (depending on extent of washing in combination with multiple aging steps). In some cases, the silica hydrogel containing low amounts of aluminium and/or sodium (on dry basis) were contacted with a solution of aluminium sulfate and/or sodium carbonate respectively before drying to adjust aluminium and/or sodium to desired levels.

### 2. Catalyst Preparation

In all cases the silica gel beads with size 2-5 mm were pre-dried to a loss of drying (LOD) <0.5 wt. %, measured at 120° C., before use. The following is a general description of making the catalyst on a basis of using 100 g silica support on dry basis. Broadly, the tantalum precursor was added to the silica via the incipient wetness impregnation method.

For every 100 g (dry basis) of silica gel support, a stabilized tantalum precursor solution was made by mixing approximately 5-6 g of tantalum precursor, such as 5.7 g tantalum ethoxide with 2-3 g, such as 2.8 g of 2,4-pentanedione (acetyl acetone). In general, 8.5 g of the stabilized tantalum precursor solution was dissolved in 65-76 g isopropanol, which was then added on to the pre-dried silica gel beads. The amount of isopropanol was adjusted based on the support pore volume, so that the solution was contained only in the silica pores, and there was no free solution outside the pores. Impregnation took around 15-40 minutes. The impregnated silica gel was kept in a sealed container for at least 1 hour before the solvent was evaporated by heating at atmospheric pressure or under vacuum. The dried material was then calcined up to 550° C. for 4 hours in air to give the finished catalyst with approximately 3.0 wt. % Ta₂O₅. In one embodiment, Catalyst A was made using this preparation method.

### Preparation of Catalyst B:

Silica gel beads with size 2-5 mm were pre-dried to a loss of drying (LOD)<0.5 wt. %, measured at 120° C., before use. For 100 g (dry basis) of silica gel support, a stabilized tantalum precursor solution was made by mixing 5.7 g tantalum ethoxide with 2.8 g of 2,4-pentanedione (acetyl acetone). 8.5 g of the stabilized tantalum precursor solution was dissolved in 73 g isopropanol, which was then added on to the pre-dried silica gel beads. Impregnation took around 15-40 minutes. The impregnated silica gel was kept in a sealed container for at least 1 hour before the solvent was evaporated by heating at atmospheric pressure. The dried material was then calcined up to 550° C. for 4 hours in air to give the finished catalyst with 2.9 wt. % Ta₂O₅, 372 ppm Na and 8 ppm Al.

The Na and Al can be assumed to be present in the support since no substantial quantities of Na or Al are present in the Ta-ethoxide, acetyl acetone or isopropanol. The amount of Na or Al in the support and catalyst is then related by the formula:

$$Support [ M ] = Catalyst [ M ] / ( 1 - Catalyst [ {Ta}_{2} {O}_{5} ] wt . \% ) , with M = Na or Al$$

Consequently, the Na and Al in the support are calculated to be 383 ppm and 8.2 ppm respectively.

### Preparation of Catalyst C:

Silica gel beads with size 2-5 mm were pre-dried to a loss of drying (LOD)<0.5 wt. %, measured at 120° C., before use. For 100 g (dry basis) of silica gel support, a stabilized tantalum precursor solution was made by mixing 5.7 g tantalum ethoxide with 2.8 g of 2,4-pentanedione (acetyl acetone). 8.5 g of the stabilized tantalum precursor solution was dissolved in 70 g isopropanol, which was then added on to the pre-dried silica gel beads.

Impregnation took around 15-40 minutes. The impregnated silica gel was kept in a sealed container for at least 1 hour before the solvent was evaporated by heating at atmospheric pressure. The dried material was then calcined up to 550° C. for 4 hours in air to give the finished catalyst with 3.3 wt. % Ta₂O₅, 392 ppm Na and 230 ppm Al.

The Na and Al can be assumed to be present in the support since no substantial quantities of Na or Al are present in the Ta-ethoxide, acetyl acetone or isopropanol. The amount of Na or Al in the support and catalyst is then related by the formula:

$$Support [ M ] = Catalyst [ M ] / ( 1 - Catalyst [ Ta 2 O 5 ] wt . \% ) , with M = Na or Al$$

Consequently, the Na and Al in the support are calculated to be 405 ppm and 238 ppm respectively.

TABLE 1
Data on Catalysts A, B and C

| | Catalyst | Catalyst | Catalyst |
|---|---|---|---|
| Sample Type | A | B | C |
| Ta [wt %] | 2.56 | 2.37 | 2.71 |
| Ta₂O₅ [wt %] | 3.1 | 2.9 | 3.3 |
| Na [ppm] | 31 | 372 | 392 |
| Al [ppm] | 6 | 8 | 230 |
| BET SSA [m²/g] | 252 | 271 | 252 |
| PV [cm³/g] | 0.95 | 1.02 | 1.01 |
| APD [Å] | 151 | 150 | 160 |

### 3. Sodium and Aluminium Analysis Method

The levels of sodium and aluminium in the catalyst compositions were measured by Atomic Absorption Spectroscopy (AA) using a Perkin-Elmer PinAAcle™900F Spectrometer and Inductively Coupled Plasma (“ICP”) Spectroscopy using a Perkin Elmer Optima 8300 ICP-OES spectrometer, respectively. Samples of catalyst were digested with hydrofluoric acid (HF). The resulting silicon tetrafluoride (SiF₄) was fumed away and the residue was analyzed for sodium and aluminium. Sodium and aluminium levels are reported as the parts per million of the catalyst after drying at 120° C. The sodium and aluminium amounts of the support and the tantalum starting material, respectively, can be determined accordingly if desired.

### 4. Tantalum Analysis Method

The levels of tantalum in the catalyst compositions were measured by Inductively Coupled Plasma (“ICP”) Spectroscopy using a Perkin Elmer Optima 8300 ICP-OES spectrometer. Samples of catalyst were digested with hydrofluoric acid (HF). The resulting silicon tetrafluoride (SiF₄) was fumed away and the residue was analyzed for tantalum. Results are reported on dried weight basis of the catalyst calcined at 500 to 550° C.

### 5. Catalytic Tests

40 grams of the catalysts synthesized according to the above procedure were placed into a respective continuous flow-operated stainless steel reactor. The reactor had initially been heated to 350° C., at a nitrogen flow rate of 500 ml/min. (Nitrogen was used only when heating the reactor, whereas the reaction was carried out without nitrogen flow, but solely with the indicated organic feed.) The reaction was then carried out using 94 wt. % aqueous ethanol mixed with acetaldehyde at a mass ratio of 2.5:1 as a feed (the mass portion of 2.5 for the 94 wt. % aqueous ethanol relates to the combined weight of water and ethanol), with a weight hourly space velocity (WHSV) of 5.0 h⁻¹ and at a pressure of 1.8 barg. The composition of the effluent was regularly monitored by an online gas chromatograph equipped with a flame-ionization detector coupled with a mass spectrometer (GC/MS).

Catalysts lose their activity for the production of 1,3-butadiene during the operation and require regeneration. Catalyst regeneration was carried out after 110 hours (h) time on stream (TOS) in situ in the stainless steel reactor, in the following four stages.
1. Desorption and removal of organic vapors
   Organic vapors were removed by purging with a stream of nitrogen (gas hourly space velocity (GHSV)=300 h⁻¹) at 350° C. for 5 hours.
2. Preliminary combustion of carbon deposits
   Deposits were burnt in a stream of air diluted by steam (GHSV=300 h⁻¹) for 15 hours. The oxygen content in the regeneration mixture (air/steam) was gradually increased from 1 to 6 vol. %, so that the temperature in the reactor would not exceed 400° C.
3. Combustion of carbon deposits

The temperature of the reactor was increased to 520° C. Deposits were finally burnt in a stream of air diluted by nitrogen (GHSV=300 h⁻¹) for 20 hours. The oxygen content in the regeneration mixture (air/nitrogen) was 6 vol. %.
4. Cooling down
   The reactor was cooled down to 350° C., in a nitrogen flow (GHSV=300 h⁻¹).

Total conversion, selectivity, yield, and productivity were calculated as shown below (EtOH=ethanol; AcH=acetaldehyde):

$$Total Conversion = \frac{moles of converted EtOH and AcH}{moles of EtOH and AcH in the feed} \cdot 100$$

$$Selectivity = \frac{C moles in 1 , 3 - butadiene}{C moles in all products} \cdot 100$$

$$Yield = \frac{Total Conversion \cdot Selectivity}{100}$$

$$Productivity = \frac{mass flow rate of 1 , 3 - butadiene}{mass of catalyst}$$

TABLE 2
Overview over physico-chemical properties of the fresh (i.e. non-regenerated) catalysts synthesized according
to the above procedure and their performance in 1,3-butadiene synthesis according to example 4

| | | | | | | | | | Productivity of |
|---|---|---|---|---|---|---|---|---|---|
| | | | | | | Total | Selectivity to | | 1,3-BDN |
| | | | | | | Conversion [%]^{b} | 1,3-BDN [%]^{b} | Yield [%]^{b} | [g_{1,3-BDN}/(g_{cat} · h)]^{b} |
| | BET | | Al | Na | | (gain/loss in | (gain/loss in | (gain/loss in | (gain/loss in |
| | SSA | APD | content | content | TOS | comparison to | comparison to | comparison to | comparison to |
| Catalyst | [m²/g] | [Å] | [ppm] | [ppm] | [h] | reference) | reference) | reference) | reference) |
| A^{a} | 252 | 151 | 6 | 31 | 100 | 26.3 | 71.3 | 18.8 | 0.59 |
| B | 271 | 150 | 8 | 372 | 100 | 27.7 | 65.8 | 18.2 | 0.58 |
| | | | | | | (+5%) | (−8%) | (−3%) | (−2%) |
| C | 252 | 160 | 230 | 392 | 100 | 27.6 | 69.1 | 19.1 | 0.60 |
| | | | | | | (+5%) | (−3%) | (+2%) | (+2%) |

(^{a}reference; ^{b}in average for a given time on stream; BET SSA—BET specific surface area; APD—average pore diameter; Al—aluminium; Na—sodium; TOS—time on stream; EtOH—ethanol; AcH—acetaldehyde; 1,3-BDN—1,3-butadiene; g—grams; h—hour; cat—catalyst; process conditions: catalysts Ta₂O₅—SiO₂ (ca. 2.5 wt. % of tantalum as Ta, dry basis; 3.1 wt % as Ta₂O₅), 94 wt. % EtOH:AcH = 2.5:1 wt./wt.; WHSV 5 h⁻¹; 350° C., 1.8 barg)

The results of the catalytic test of catalyst B (not according to the invention) in Table 2 show that an increase in sodium level increases the total conversion compared to ultra pure catalyst A (not according to the invention), however, it has a strongly detrimental effect on the selectivity to 1,3-butadiene. Thus, both the achieved yield and productivity of 1,3-butadiene are lower for catalyst B compared to catalyst A.

The results of the catalytic test of catalyst C (according to the invention) in Table 2 demonstrate that a simultaneous increase in aluminium level surprisingly offsets the detrimental effects of the increased sodium level. In the catalytic test of catalyst C not only the total conversion was increased, but also the selectivity loss to 1,3-butadiene was small, so that overall an increase in both yield and productivity of 1,3-butadiene was advantageously achieved with the catalyst according to the invention.

1. A supported catalyst comprising
(i) a support, and
(ii) 0.1 to 10 wt % of tantalum, calculated as Ta₂O₅ and based on the total weight of the catalyst,
wherein the supported catalyst further comprises from 50 to 350 ppm of aluminium, based on the total weight of the catalyst, and from 300 to 500 ppm of sodium, based on the total weight of the catalyst.

2. The supported catalyst according to claim 1, wherein the support comprises one or more of ordered and non-ordered porous silica supports, other porous oxide supports and mixtures thereof, preferably from ZrO₂, TiO₂, MgO, ZnO, NiO, and CeO₂.

3. The supported catalyst according to claim 1, wherein the supported catalyst has a BET specific surface area in a range of from 130-550 m²/g, preferably in a range of from 190 to 280 m²/g.

4. The supported catalyst according to claim 1, wherein the weight ratio of aluminium to sodium is in a range of from 0.1 to 1.2, preferably 0.2 to 1.0.

5. A catalyst reaction tube for the production of 1,3-butadiene comprising at least one packing of the supported catalyst as defined in claim 1 and one or more packings of inert material.

6. A reactor for the production of 1,3-butadiene comprising one or more of the catalyst reaction tubes as defined in claim 5.

7. A plant for the production of 1,3-butadiene comprising one or more of the reactors as defined in claim 6, and means for regenerating the supported catalyst in said one or more reactors,
preferably wherein the plant also comprises an acetaldehyde-producing pre-reactor with one or more reaction tubes comprising a supported or unsupported (bulk) catalyst comprising one or more of zinc, copper, silver, chromium, magnesium and nickel.

8. A process for the production of 1,3-butadiene, the process comprising
(i) contacting a feed comprising ethanol and acetaldehyde with the supported catalyst as defined in claim 1 to obtain a raw product comprising 1,3-butadiene.

9. The process according to claim 8, wherein the (i) contacting takes place at a temperature in a range of from 200 to 500° C., preferably from 250 to 450° C., more preferably from 300 to 400° C.

10. The process according to claim 8, wherein the (i) contacting takes place at a weight hourly space velocity in a range of from 0.2 to 10 h⁻¹, preferably from 1 to 7 h⁻1.

11. The process according to claim 8, wherein the (i) contacting takes place at a pressure in a range of from 0 to 10 barg, preferably from 1 to 3 barg.

12. The process according to claim 8, further comprising
(ii) separating the raw product at least into a first portion comprising 1,3-butadiene, a second portion comprising acetaldehyde and a third portion comprising ethanol, preferably wherein at least part of the second, of the third, or of both the second and of the third portions is recycled into the feed.

13. The process of claim 8, wherein the (i) contacting takes place in a continuous flow of the feed in a reactor.

14. A process for the production of the supported catalyst as defined in claim 1 comprising or consisting of the following steps:
(i) impregnation of the support with aluminium and sodium levels defined by the formulas below based on the weight of the catalyst support, with a solution of a tantalum precursor, to form a supported tantalum catalyst precursor,
wherein the lower limit is defined by: Support [M]_{LL}=Catalyst [M]_{LL}/(1−Catalyst [Ta₂O₅] wt. %), with M=Na or Al; where Catalyst [Na]_{LL}=300 ppm and Catalyst [Al]_{LL}=50 ppm; and
the upper limit is defined by: Support [M]_{UL}=Catalyst [M]_{UL}/(1−Catalyst [Ta₂O₅] wt. %), with M=Na or Al; where Catalyst [Na]_{UL}=500 ppm and Catalyst [Al]_{UL}=350 ppm;
(ii) drying the supported tantalum catalyst precursor, and
(iii) calcining the dried supported tantalum catalyst precursor, to form a supported tantalum catalyst.

15. The process for the production of the supported catalyst as defined in claim 14, wherein the supported catalyst is a silica supported catalyst and the method comprises or consists of:
(i) reacting an aqueous silicate, preferably sodium silicate, solution with an acid to form a hydrosol,
(ii) dispersion and gelation of the hydrosol to form hydrogel beads,
(iii) one or more optional additional steps of (pre-) aging, acidification, washing and pH adjustment,
a. aging of the hydrogel beads at temperature T1,
b. acidification of the aged hydrogel beads,
c. washing, preferably with water that is deionized and acidified to pH 3-4, of the acidified aged hydrogel beads,
d. adjusting the pH of the washed hydrogel beads obtained in step (c), preferably to a pH of about 8-10,
(iv) aging of the hydrogel beads at temperature T2, with T2>T1,
(v) acidification of the aged hydrogel beads,
(vi) washing, preferably with water that is deionized and acidified to pH 3-4, of the acidified aged hydrogel beads,
(vii) optionally adjusting the pH of the washed hydrogel beads obtained in step (vi),
(viii) drying the washed hydrogel beads obtained in step (vi) or (vii) to obtain a silica support,
(ix) optionally, sieving of the silica support obtained in step (viii),
(x) impregnation of the silica support obtained in step (viii) or (ix) with a solution of a tantalum precursor, to form a supported tantalum catalyst precursor,
(xi) drying the supported tantalum catalyst precursor, and
(xii) calcining the dried supported tantalum catalyst precursor, to form a supported tantalum catalyst.

16. Use of the supported catalyst as defined in claim 1 for the production of 1,3-butadiene from a feed comprising ethanol and acetaldehyde, preferably for increasing the yield of 1,3-butadiene.

17. Use of aluminium in an amount in a range of from 50 to 350 ppm, based on the total weight of the catalyst, in a supported catalyst for the production of 1,3-butadiene from a feed comprising ethanol and acetaldehyde, the catalyst comprising
a support,
300 to 500 ppm of sodium, based on the total weight of the catalyst, and
0.1 to 10 wt. % of tantalum, calculated as Ta₂O₅ and based on the total weight of the catalyst,
for increasing the yield of 1,3-butadiene.
